(19) United States
(12) Patent Application Publication
(10) Pub. No.: US 2004/0224019 A1
(43) Pub. Date: Nov. 11, 2004

(54) Oral controlled release system for targeted drug delivery into the cell and its nucleus for gene therapy, DNA vaccination, and administration of gene based drugs

(76) Inventors: Adi Shefer, East Brunswick, NJ (US); Samuel Shefer, East Brunswick, NJ (US)

Correspondence Address: Diane Dunn McKay, Esq., Mathews, Collins, Shepherd & McKay, P.A., Suite 306, 100 Thanet Circle, Princeton, NJ, 08540, US

(21) Appl. No.: 10/791,989

(22) Filed: Mar. 3, 2004

Related U.S. Application Data
(63) Continuation-in-part of application No. 10/315,801, filed on Dec. 9, 2002.

(51) Int. Cl. A61K 9/26

(52) U.S. Cl. 424/469

(57) ABSTRACT

The present invention relates to an oral drug delivery system which delivers pharmaceutical active ingredients into the cell and/or its nucleus for the effective administration of nucleic acids including gene therapy, vaccination, administration of gene based drugs or administration of gene based treatment modalities, including the use of sense, antisense nucleotide sequences, antigens, antibodies, ribozymes, as well as oligonucleotides and polynucleotide constructs for gene correction. These actives may also include viruses, vectors, proteins, peptides, and nucleic acids, DNA or RNA fragments, which code functionally active or inactive or conditionally inactivatable proteins. The controlled delivery system of the present invention is substantially a free-flowing powder consisting of solid hydrophobic nanospheres encapsulated in pH sensitive microspheres. The controlled release system can be used to target and control the release of pharmaceutical active ingredients onto certain regions of the gastrointestinal tract, specially the small intestine. The invention further pertains to pharmaceutical products comprising the controlled release system of the present invention.

### CROSS REFERENCE TO RELATED APPLICATION

This application is a continuation-in-part of U.S. patent application Ser. No. 10/315,801, filed Dec. 9, 2002, entitled pH Triggered Targeted Controlled Release Systems for the Delivery of Pharmaceutical Active Ingredients, entitled pH Triggered Site Specific Targeted Controlled Drug Delivery System for the Treatment of Cancer. The entirety of each of these pending applications is hereby incorporated by reference into this application.

### BACKGROUND OF THE INVENTION

1. Field of the Invention

The present invention relates to an oral drug delivery system which delivers pharmaceutical active ingredients into the cell and/or its nucleus for nucleic acid or gene therapy, DNA vaccination, for example, administration of gene based drugs or gene based treatment modalities, including the use of sense, antisense nucleotide sequences, ribozymes, chimeric oligonucleotides constructs for gene correction, nucleic acids which encode antigens or antibodies or functional portions thereof. These active ingredients may also include viruses, vectors, proteins, peptides, and nucleic acids, DNA or RNA fragments, which code functionally active or inactive or conditionally inactivatable proteins. More specifically, the present invention relates to a method using solid hydrophobic nanospheres encapsulated in a pH sensitive microsphere for oral delivery of pharmaceutical active ingredients, including nucleic acids, oligonucleotides, polynucleotides, plasmid DNA, RNA, proteins, and gene based drugs, into a target cell and/or its nucleus. The novel aspect of the invention is the administration of pharmaceutical active ingredients into the cell and/or its nucleus by the oral route.

The invention further relates to pharmaceutical formulations that are safe for oral ingestion in various forms, including tablet, capsule, liquid, elixir, powder, granules, and the like, comprising the targeted controlled delivery system of the present invention. The targeted controlled delivery system of the present invention may also be admixed with food or drinks and it may be self-administered for added convenience rather than requiring the expense of a health professional for administration as is often required for injections and other inoculations.

2. Description of the Related Art

The conventional route of vaccination and gene therapy is by injection. This is primarily due to the lack of absorption of genetic based drugs through the gastrointestinal (GI) tract. However, injections are painful and sometimes difficult to administer relative to other dosage forms. Oral delivery of genetic based drugs has been the subject of major research efforts within the pharmaceutical industry. Oral delivery is preferable to injections for patient acceptance since it is less painful and more convenient for the patient. However, delivery of gene based drugs through the gastrointestinal tract has a number of problems such as low pH in the stomach, proteolytic degradation of the drug in the small intestine, low absorption through the intestinal membrane, and limited stability of such formulations, especially as an aqueous solution, which are all potential barriers to absorption of genetic based drugs following oral administration.

Genetic vaccines have proven extremely efficient in eliciting immune responses against a wide variety of microbes. Protection in animal models has been demonstrated among others for influenza virus, malaria, bovine herpes virus, rabies virus, papilloma virus, herpes simplex virus, mycoplasma, and lymphocytic choriomeningitis [J. J. Donnelly, J. B. Ulmer, J. W. Shiver and M. A. Liu., Annual Review of Immunology 15, pp. 617-648, 1997; J. B. Ulmer, R. R. Deck, C. M. Dewitt, J. I. Donnelly and M. A. Liu, Immunology 89, pp. 59-67, 1996]. Because antigen expression is maintained over a period of time, single dose immunization may become a reality. Genetic vaccines potentially can significantly lower the cost per dose.

The oral route for vaccination offers significant advantage in that it reduces labor costs, is time-saving, decreases the possibilities for cross-contamination with needles and does not involve inventory handling. The preferred transfection site for oral genetic based drugs in the small intestine, thus being able to retain these types of genetic based drugs until they reach the small intestine is essential to the successful development of oral nucleic acid, e.g., DNA vaccination, oral administration of gene based drugs, or oral administration of gene based treatment modalities, including the use of sense, antisense nucleic acids, including but not limited to those which encode antigens and antibodies, ribozymes, as well as chimeric oligonucleotides constructs for gene correction.

pH sensitive materials have been widely used as enteric coatings to encapsulate and protect active ingredients during transit through the stomach, and then release the agent shortly after entering the small intestine. pH-sensitive coatings that achieve delivery in the colon have been described in patents such as U.S. Pat. No. 4,910,021 and WO 9001329. U.S. Pat. No. 4,910,021 describes using a pH-sensitive material to coat a capsule. WO 9001329 describes using pH-sensitive coatings on beads containing acid, where the acid in the bead core prolongs dissolution of the pH-sensitive coating.

U.S. Pat. No. 6,068,859 discloses a controlled-release dosage form of azithromycin having an improved side effect profile; a process for preparing the dosage form; and a method of treating a microbial infection, comprising administering azithromycin in such a controlled-release dosage form to a mammal, including a human patient, in need of such treatment. A first delayed release embodiment according to the invention is a pH-dependent coated tablet which comprises a tablet core comprising azithromycin, a disintegrant, a lubricant, and one or more pharmaceutical carriers, such core being coated with a material, preferably a polymer, which is substantially insoluble and impermeable at the pH of the stomach, and which is more soluble and permeable at the pH of the small intestine. Preferably, the coating polymer is substantially insoluble and impermeable at pH<5.0, and water-soluble at pH>5.0.

U.S. Pat. No. 6,228,396 discloses a colonic drug delivery composition, i.e., a starch capsule containing a drug, the starch capsule being provided with a coating such that the drug will only be released from the capsule in the colon. The coating is contemplated to be a pH sensitive material, a redox sensitive material, or a material broken down by specific enzymes or bacteria present in the colon. The drug to be delivered may be one for local action in the colon or a systemically active drug to be absorbed from the colon.

U.S. Pat. No. 5,175,003 discloses a dual mechanism polymer mixture composed of pH-sensitive enteric materials and film-forming plasticizers capable of conferring permeability to the enteric material, for use in drug-delivery systems; a matrix pellet composed of a dual mechanism polymer mixture permeated with a drug and sometimes covering a pharmaceutically neutral nucleus; a membrane-coated pellet comprising a matrix pellet coated with a dual mechanism polymer mixture envelope of the same or different composition; and a pharmaceutical dosage form containing matrix pellets. The matrix pellet releases acid-soluble drugs by diffusion in acid pH and by disintegration at pH levels of nominally about 5.0 or higher.

U.S. Pat. No. 6,602,524 discloses methods for treating tumors comprising the administration of drug loaded pH-sensitive microspheres to a patient in need of anticancer therapy. The pH-sensitive microspheres have a swelling transition with the pH range found in or near tumor tissue. When the microspheres swell, the loaded drug is released into the microenvironment of the tumor tissue. The microspheres are capable of effectively releasing a loaded substance at a pre-determined pH.

U.S. Pat. No. 4,503,030 discloses an osmotic device for dispensing a drug to certain pH regions of the gastrointestinal tract. More particularly, the invention relates to an osmotic device comprising a wall formed of a semi-permeable pH sensitive composition that surrounds a compartment containing a drug, with a passageway through the wall connecting the exterior of the device with the compartment. The device delivers the drug at a controlled rate in the region of the gastrointestinal tract having a pH of less than 3.5, and the device self-destructs and releases all its drug in the region of the gastrointestinal tract having a pH greater than 3.5, thereby providing total availability for drug absorption.

The major weakness of all the above systems is that the size of these pharmaceutical ingredients is small enough to diffuse and release prematurely, before the systems reached the target site or the desired pH environment. In order to overcome premature release the pharmaceutical active ingredients need to be incorporated into larger structures that will be entrapped and retained within the pH sensitive matrix structure until the systems reaches the target site or pH environment.

U.S. Pat. Nos. 5,609,590 and 5,358,502 disclose an osmotic bursting device for dispensing a beneficial agent to an aqueous environment. The device comprises a beneficial agent and osmagent surrounded at least in part by a semi-permeable membrane. Alternatively the beneficial agent may also function as the osmagent. The semi-permeable membrane is permeable to water and substantially impermeable to the beneficial agent and osmagent. A trigger means is attached to the semi-permeable membrane (e.g., joins two capsule halves). The trigger means is activated by a pH of from 3 to 9 and triggers the eventual, but sudden, delivery of the beneficial agent. These devices enable the pH-triggered release of the beneficial agent core as a bolus by osmotic bursting.

U.S. Pat. No. 5,316,774 discloses a composition for the controlled release of an active substance comprising a polymeric particle matrix, where each particle defines a network of internal pores. The active substance is entrapped within the pore network together with a blocking agent having physical and chemical characteristics selected to modify the release rate of the active substance from the internal pore network. In an exemplary embodiment, drugs may be selectively delivered to the intestines using an enteric material as the blocking agent. The enteric material remains intact in the stomach but degrades under the pH conditions of the intestines. In another exemplary embodiment, the sustained release formulation employs a blocking agent, which remains stable under the expected conditions of the environment to which the active substance is to be released. The use of pH-sensitive materials alone to achieve site-specific delivery is difficult because of leaking of the beneficial agent prior to the release site or desired delivery time and it is difficult to achieve long time lags before release of the active ingredient after exposure to high pH (because of rapid dissolution or degradation of the pH-sensitive materials).

There are also hybrid systems which combine pH-sensitive materials and osmotic delivery systems. These devices provide delayed initiation of sustained-release of the beneficial agent. In one device a pH-sensitive matrix or coating dissolves releasing osmotic devices that provide sustained release of the beneficial agent see U.S. Pat. Nos. 4,578,075, 4,681,583, and 4,851,231. A second device consists of a semipermeable coating made of a polymer blend of an insoluble and a pH-sensitive material. As the pH increases, the permeability of the coating increases, increasing the rate of release of beneficial agent see U.S. Pat. Nos. 4,096,238, 4,503,030, 4,522,625, and 4,587,117.

U.S. Pat. No. 5,484,610 discloses terpolymers, which are sensitive to pH and temperature and which are useful carriers for conducting bioactive agents through the gastric juices of the stomach in a protected form. The terpolymers swell at the higher physiologic pH of the intestinal tract causing release of the bioactive agents into the intestine. The terpolymers are linear and are made up of 35 to 99 wt % of a temperature sensitive component, which imparts to the terpolymer LCST (lower critical solution temperature) properties below body temperatures, 1 to 30 wt % of a pH sensitive component having a pKa in the range of from 2 to 8 which functions through ionization or deionization of carboxylic acid groups to prevent the bioactive agent from being lost at low pH but allows bioactive agent release at physiological pH of about 7.4 and a hydrophobic component which stabilizes the LCST below body temperatures and compensates for bioactive agent effects on the terpolymers. The terpolymers provide for safe bioactive agent loading, a simple procedure for dosage form fabrication and the terpolymer functions as a protective carrier in the acidic environment of the stomach and also protects the bioactive agents from digestive enzymes until the bioactive agent is released in the intestinal tract.

U.S. Pat. No. 6,103,865 discloses pH-sensitive polymers containing sulfonamide groups, which can be changed in physical properties, such as swellability and solubility, depending on pH and which can be applied for a drug-delivery system, bio-material, sensor, and the like, and a preparation method therefore. The pH-sensitive polymers are prepared by introduction of sulfonamide groups, various in pKa, to hydrophilic groups of polymers either through coupling to the hydrophilic groups of polymers, such as acrylamide, N,N-dimethylacrylamide, acrylic acid, N-isopropylacrylamide and the like or copolymerization with other polymerizable monomers. These pH-sensitive polymers may have a structure of linear polymer, grafted copolymer, hydrogel or interpenetrating network polymer.

U.S. Pat. No. 5,656,292 discloses a composition for pH dependent or pH regulated controlled release of active ingredients especially drugs. The composition consists of a compactable mixture of the active ingredient and starch molecules substituted with acetate and dicarboxylate residues. The preferred dicarboxylate acid is succinate. The average substitution degree of the acetate residue is at least 1 and 0.2-1.2 for the dicarboxylate residue. The starch molecules can have the acetate and dicarboxylate residues attached to the same starch molecule backbone or attached to separate starch molecule backbones. The invention also discloses methods for preparing said starch acetate dicarboxylates by transesterification or mixing of starch acetates and starch dicarboxylates respectively.

U.S. Pat. Nos. 5,554,147, 5,788,687, and 6,306,422 disclose a method for the controlled release of a biologically active agent wherein the agent is released from a hydrophobic, pH-sensitive polymer matrix. The polymer matrix swells when the environment reaches pH 8.5, releasing the active agent. A polymer of hydrophobic and weakly acidic comonomers is disclosed for use in the controlled release system. Also disclosed is a specific embodiment in which the controlled release system may be used. The pH-sensitive polymer is coated onto a latex catheter used in ureteral catheterization. A ureteral catheter coated with a pH-sensitive polymer having an antibiotic or urease inhibitor trapped within its matrix will release the active agent when exposed to high pH urine.

It is well documented in the literature that the pH values of human tumors are substantially and consistently lower than that in normal tissue (Gerweck L. E. et al., “Cellular pH gradient in tumor versus normal tissue: potential exploitation for the treatment of cancer,” Cancer Research 56, Issue 6, pp. 1194-1198, 1996). Deborah M. P. et al. (“The Relationship between Intracellular and Extracellular pH in Spontaneous Canine Tumors,” Clinical Cancer Research Vol. 6, pp. 2501-2505, 2000) have also reported that the cellular uptake of chemotherapeutic drugs may be dependent on the pH gradient between the intracellular and extracellular compartments. Hence, pH-sensitive delivery systems can be a useful route for tumor targeting. The pH difference provides an exploitable avenue for targeting the chemotherapeutic agent to the tumor site. Also, weakly acidic drug delivery systems, which are relatively lipid soluble in their nonionized state have been found to diffuse freely across the cell membrane and, upon entering a relatively basic intracellular compartment, become trapped and accumulate within a cell, leading to substantial differences in the intracellular/extracellular drug distribution between tumor and normal tissue for drugs exhibiting appropriate pKas.

U.S. Pat. No. 6,475,995 discloses nanoparticle coacervates of nucleic acids and polycations serve as effective vaccines when administered orally. They can induce immunity to a variety of disease causing agents and raise a protective response to allergens.

Liposomes have been widely used as delivery vehicles for anti-cancer drugs. U.S. Pat. No. 6,426,086 discloses a serum of liposomes that are pH-sensitive for controlled drug delivery. The liposomes are complexed with a molecule comprising a thermally-sensitive polymer showing lower critical solution temperature behavior in aqueous solutions, said thermally-sensitive polymer bearing a hydrophobic substituent and a pH sensitive substituent, wherein said hydrophobic substituent is less than 10 kD and which pH sensitive substituent remains ionizable following said covalent bonding to said thermally-sensitive polymer, and whose pH sensitive does not depend on cleavage of the covalent bond to said thermally-sensitive polymer. The limited stability of liposomes both in terms of shelf life and after administration, their ability to encapsulate only certain types of molecules, as well as their rapid clearance from the blood have hampered the use of liposomes as effective controlled drug delivery systems.

U.S. Pat. No. 6,602,524 discloses methods for treating tumors comprising the administration of a drug loaded in pH-sensitive microspheres wherein said pH-sensitive microspheres comprise a cross-linked polymer gel comprising ethyl methacrylate, diethyl aminoethyl methacrylate, and divinyl benzene. The pH-sensitive microspheres have a swelling transition with the pH range found in or near tumor tissue. When the microspheres swell, the loaded drug is released into the microenvironment of the tumor tissue. The microspheres are capable of effectively releasing a loaded substance at a pre-determined pH. The major drawback of the pH sensitive microspheres desclosed in U.S. Pat. No. 6,602,524 is that the matrix structure created by the cross-linking of ethyl methacrylate, diethyl aminoethyl methacrylate, and divinyl benzene is most likely to facilitate the diffusion and premature release of the chemotherapeutic agents, that are relatively small molecules, before these microspheres reach the target site of the tumor. Further protection of these molecules is needed to ensure that they are sustained by microspheres until they reach the tumor site. Also, at a pre-determined pH these microspheres quickly release the drug and cannot provide prolonged release of these active agents over an extended period of time.

U.S. Pat. No. 6,365,187 discloses bioadhesive polymers in the form of, or as a coating on, microcapsules containing drugs or bioactive substances which may serve for therapeutic, or diagnostic purposes in diseases of the gastrointestinal tract, are described. The polymeric microspheres all have a bioadhesive force of at least 11 mN/cm2 (110 N/m2) Techniques for the fabrication of bioadhesive microspheres, as well as a method for measuring bioadhesive forces between microspheres and selected segments of the gastrointestinal tract in vitro are also described. This quantitative method provides a means to establish a correlation between the chemical nature, the surface morphology and the dimensions of drug-loaded microspheres on one hand and bioadhesive forces on the other, allowing the screening of the most promising materials from a relatively large group of natural and synthetic polymers which, from theoretical consideration, should be used for making bioadhesive microspheres.

Several modifications of DNA-cation particles have been disclosed to circumvent the nonspecific interactions of the DNA-cation particle and the toxicity of cationic particles. Examples of these modifications include attachment of steric stabilizers, e.g. polyethylene glycol, which inhibit nonspecific interactions between the cation and biological polyanions. Another example is recharging the DNA particle by the additions of polyanions which interact with the cationic particle, thereby lowering its surface charge, i.e., recharging of the DNA particle, U.S. application Ser. No. 09/328,975. Another example is cross-linking the polymers and thereby caging the complex U.S. application Ser. No. 08/778,657, U.S. application Ser. No. 09/000,692, U.S. Application Ser. No. 97/24089, U.S. application Ser. No. 09/070,299, and U.S. application Ser. No. 09/464,871. Nucleic acid particles can be formed by the formation of chemical bonds and template polymerization U.S. application Ser. No. 08/778,657, U.S. application Ser. No. 09/000,692, U.S. Application Ser. No. 97/24089, U.S. application Ser. No. 09/070,299, and U.S. application Ser. No. 09/464,871. The drawback of these modifications is that they are most likely irreversible rendering the particle unable to interact with the cell to be transfected, and/or incapable of escaping from the lysosome once taken into a cell, and/or incapable of entering the nucleus once inside the cell.

U.S. Pat. No. 6,383,811 discloses the use of a polyampholyte in a condensed polynucleotide complex for purposes of nucleic acid delivery to a cell. The complex can be formed with an appropriate amount of positive and/or negative charge such that the resulting complex can be delivered to the extravascular space and may be further delivered to a cell.

U.S. Patent Application Publication No. 20030026841 discloses compositions and methods for drug delivery using pH sensitive molecules. A polyampholyte is utilized in a condensed polynucleotide complex for purposes of nucleic acid delivery to a cell. The complex can be formed with an appropriate amount of positive and/or negative charge such that the resulting complex can be delivered to the extravascular space and may be further delivered to a cell.

The prior art of which applicant is aware, however, does not set forth a targeted controlled delivery system of pharmaceutical active ingredients into the cell and/or its nucleus that consists of solid hydrophobic nanospheres encapsulated in a pH sensitive microsphere. The present invention overcome the weaknesses of the systems disclosed in the prior art and addresses the ongoing need for an effective delivery system for gene therapy, DNA vaccination, administration of gene based drugs.

Gene therapy and nucleic acid vaccination are the most promising areas of research today. It would therefore be extremely useful if one had an effective way to introduce nucleic acids and/or genes into cells that yield long-term expression. It is therefore desirable to provide a method for the oral administration of pharmaceutical active ingredients into the cell and/or its nucleus for gene therapy, oral vaccination by means of nucleic acids, oral administration of gene based drugs or oral administration of nucleic acid based treatment modalities, including the use of sense, antisense nucleic acids, antigens, antibodies, ribozymes, as well as chimeric oligonucleotides constructs for gene correction. These actives may also include DNA or RNA fragments, which code functionally active or inactive or conditionally inactivatable proteins. It is further desirable to provide effective pharmaceutical formulations comprising gene based drugs that are safe for oral ingestion in various forms, including tablet, capsule, liquid, elixir, powder, granules, and the like for added convenience and reduce the expense of a health professional.

### SUMMARY OF THE INVENTION

The present invention relates to an oral drug delivery system which delivers pharmaceutical active ingredients into the cell and/or its nucleus for nucleic acid/gene therapy, vaccination, administration of gene based drugs or administration of nucleic acid based treatment modalities, including the use of sense, antisense nucleotide sequences, antigens, antibodies, ribozymes, as well as oligonucleotides constructs for gene correction. These actives may also include viruses, vectors, proteins, peptides, and nucleic acids, DNA or RNA fragments, which code functionally active or inactive or conditionally inactivatable proteins. More specifically, the present invention relates to a method using solid hydrophobic nanospheres encapsulated in a pH sensitive microsphere for oral delivery of pharmaceutical active nucleic acids including but not limited to oligonucleotides, oligoribonucleotides, polynucleotides, polyribonucleotides, genes including pharmacologically effective fragments thereof, messenger ribonucleic acids (mRNA), and ribozymes into the cytoplasm of target cells by means of oral administration. Embodiments of the present invention are employed to deliver nucleic acids as defined herein across nuclear membranes, i.e., to the nucleus, of target cells. The novel aspect of the invention includes the administration of pharmaceutical active ingredients into the cell and/or its nucleus by the oral route.

The present invention also pertains to solid hydrophobic nanospheres encapsulated in a pH sensitive microsphere that can be loaded with a pharmaceutical active ingredient useful for gene therapy, DNA vaccination, oral administration of gene based drugs or administration of gene based treatment modalities, including the use of sense, antisense nucleotide sequences, antigens, antibodies, ribozymes, as well as chimeric oligonucleotides constructs for gene correction, by the oral route. The microspheres are capable of effectively releasing the pharmaceutical active ingredients and the nanospheres loaded the same or different pharmaceutical active ingredient at a pre-determined pH. The nanospheres enhance the bioavailability of the active ingredients and can be designed to release their pharmaceutical active ingredient over an extended period of time at the specified pH or at a pH of the small intestine.

The invention further relates to pharmaceutical formulations that are safe for oral ingestion in various forms, including tablet, capsule, liquid, elixir, powder, granules, etc., comprising the targeted controlled delivery system of the present invention. The targeted controlled delivery system of the present invention can be admixed with food or drinks and it can be self-administered for added convenience rather than requiring the expense of a health professional for administration as is often required for injections and other inoculations.

The pharmaceutical active ingredient encapsulated in the controlled release system of the present invention include, but are not limited to proteins, enzyme, peptides, polypeptide viruses, antigens, (including food allergen), nucleic acids, oligonucleotides, a polynucleotide, plasmid DNA, RNA, proteins, and gene based treatment modalities, including the use of sense, antisense nucleotide sequences, antigens, antibodies, ribozymes, as well as chimeric oligonucleotides constructs for gene correction. These actives may also include DNA or RNA fragments, which code functionally active or inactive or conditionally inactivatable proteins.

In one embodiment, the nanospheres of the present invention are bioadhesive. Bioadhesive nanospheres of the present invention can be created by incorporating a bioadhesive material into the solid hydrophobic matrix of the nanospheres, by incorporating a bioadhesive material in the pH sensitive microsphere matrix, or by using a bioadhesive material in the nanosphere matrix in conjunction with bioadhesive material in the microsphere matrix.

The oral controlled delivery system of the present invention is a free-flowing powder formed of solid hydrophobic nanospheres comprising various active ingredients including nucleic acids, for example, that are encapsulated in a pH sensitive microsphere, having the advantages of:

(i) protection of the pharmaceutical active ingredients, during storage, or until they reach the target site;

(ii) pH triggered controlled release of a first pharmaceutical active ingredient from the microspheres and of a second pharmaceutical active ingredient from the nanospheres at the predetermined pH, and,

(iii) site specific targeted delivery and enhanced deposition of the nanospheres comprising pharmaceutical active ingredients at the target region of the gastrointestinal tract;

(iv) enhanced bioavailability and efficacy of pharmaceutical active ingredients encapsulated in the nanospheres; and

(v) prolonged release of pharmaceutical active ingredients encapsulated in the nanospheres over an extended period of time.

The invention also provides a method for producing the multi component controlled release system of the present invention including active ingredients comprising the steps of:

(i) incorporating the pharmaceutical active ingredients, e.g., nucleic acids, into solid hydrophobic nanospheres;

(ii) forming an aqueous mixture comprised of one or more pharmaceutical active agents, the nanospheres, and pH sensitive materials; and

(iii) spray drying the mixture to form a dry powder composition.

The invention further provides a process for producing the multi component controlled release system of the present invention including the pharmaceutical active ingredients that comprise the steps of:

(i) heating hydrophobic materials to a temperature above the melting point of the materials to form a melt;

(ii) dissolving or dispersing a first pharmaceutical active agent into the melt;

(iii) dissolving or dispersing a second pharmaceutical active agent, pH sensitive material, and a targeting material, in the aqueous phase;

(iv) heating the composition to above the melting temperature of the hydrophobic materials;

(v) mixing the hot melt with the aqueous phase to form a dispersion;

(vi) high shear homogenization of the dispersion at a temperature above the melting temperature until a homogeneous fine dispersion is obtained having a sphere size of from about 1 micron to about 2 microns;

(vii) cooling the dispersion to ambient temperature; and

(viii) spray drying the emulsified mixed suspension to form a dry powder composition.

In an embodiment, the delivery system comprises a nucleic acid or nucleic acid-polymer complexes deliverable to the intestines. In an embodiment, the delivery system comprises nucleic acid or a stabilized nucleic acid delivered to the intestines through oral intake. In an embodiment, the delivery system comprises nucleic acid or stabilized nucleic acid that is delivered to the intestines through oral intake.

In an embodiment, the nucleic acid encodes an antigen. In an embodiment, the nucleic acid upon delivery as described herein expresses an antigen. In an embodiment, the nucleic acid expresses an antigen encoded by a cellular gene of unknown function. In an embodiment, the nucleic acid expresses an antigen derived from a pathogen, particularly a viral pathogen. In an embodiment, the nucleic acid expressed an antigen from human immunodeficiency virus, human hepatitis A virus, human hepatitis B virus, human hepatitis C virus, influenza virus, smallpox (variola) virus, or human herpes virus (type I through VIII). In an embodiment, the nucleic acid expressed an antigen from a bacterial pathogen. In an embodiment, the nucleic acid encodes a bacterial antigen, e.g., of pneumococcus, streptococcus, bacillus, E. coli or anthrax origin. In an embodiment, the present invention provides a process for delivering nucleic acids into lymphoid cells.

In an embodiment, the transferred nucleic acid expresses an antigen. In an embodiment, the expression of the antigen induces an immune response. In an embodiment, the expression of the antigen induces an antigen-specific immune response. In an embodiment, the antigen-specific immune response results in the formation of antigen-specific antibodies. In an embodiment, the antigen-specific immune response results in the formation of antigen-specific antibodies which may be obtained and purified from the blood of the host. In an embodiment, B cells may be obtained from a host which produces antigen-specific antibodies. In an embodiment, B cells may be obtained from the host which produce antigen-specific antibodies and fused with myeloma cells to create monoclonal antibody producing cells. In an embodiment, the genetic immunization results in the induction of an antigen-specific cellular immune response. In an embodiment, the genetic immunization results in the induction of antigen-specific T-cells.

In another embodiment a method is described for generating an immune response in a host. A nucleic acid is administered which encodes an antigen in an amount sufficient to induce an effective immune response directed against the expressed antigen. The nucleic acid is delivered to the intestinal lumen.

The invention also provides pharmaceutical products comprising the multi component controlled release system of the present invention.

The invention will be more fully described by reference to the following drawings:

### BRIEF DESCRIPTION OF THE DRAWINGS

FIG. 1 is a schematic diagram of the multi component controlled delivery system of the present invention that comprises solid hydrophobic nanospheres encapsulated in a pH sensitive microsphere

FIG. 2 is a schematic diagram of the release profile of active ingredients from the multi component controlled release system of the present invention.

FIG. 3 is a schematic diagram of the nanospheres encapsulated in the pH sensitive microsphere of the present invention.

### DETAILED DESCRIPTION OF THE INVENTION

The present invention relates to an oral drug delivery system which delivers pharmaceutical active ingredients into the cell and/or its nucleus for nucleic acid therapy, including gene therapy, vaccination, administration of gene based drugs or administration of gene based treatment modalities, including the use of sense, antisense nucleotide sequences, antigens, antibodies, ribozymes, as well as chimeric oligonucleotides constructs for gene correction. These actives may also include viruses, vectors, proteins, peptides, and nucleic acids, DNA or RNA fragments, which code functionally active or inactive or conditionally inactivatable proteins.

More specifically, the present invention relates to a method using solid hydrophobic nanospheres encapsulated in a pH sensitive microsphere for oral delivery of pharmaceutical active ingredients, including oligonucleotides, polynucleotides, plasmid DNA, RNA, proteins, and gene based drugs, into a target cell and/or its nucleus. The novel aspect of the invention is the administration of pharmaceutical active ingredients into the cell and/or its nucleus by the oral route.

“Pharmaceutical active agent” as used herein includes but is not limited to biological molecules including nucleic acids. “Nucleic acid” is a well-known term in the art and is used herein to include, for example, oligonucleotides, oligoribonucleotides, polynucleotides, polyribonucleotides, genes (including pharmacologically effective fragments thereof), messenger ribonucleic acids (mRNA), and ribozymes. Oligonucleotides and oligoribonucleotides correspond to DNA and RNA, respectively. Oligonucleotides and oligoribonucleotides for use in the present invention are generally oligomers within the range of size of about two (2) to about thirty five (35) bases in length. Oligonucleotides and oligoribonucleotides preferred for use in the present invention are preferably within the range of size of about two (10) to about thirty five (25) bases in length. Polynucleotides and polyribonucleotides correspond to DNA and RNA, respectively. Polynucleotides and polyribonucleotides for use in the present invention are generally nucleic acids within the range of size of about thirty five (36) bases to about one thousand two hundred (1,200) bases in length. Polynucleotides and polyribonucleotides for use in the present invention are preferably within the range of size of about thirty six (36) to about two hundred and fifty (250) bases in length.

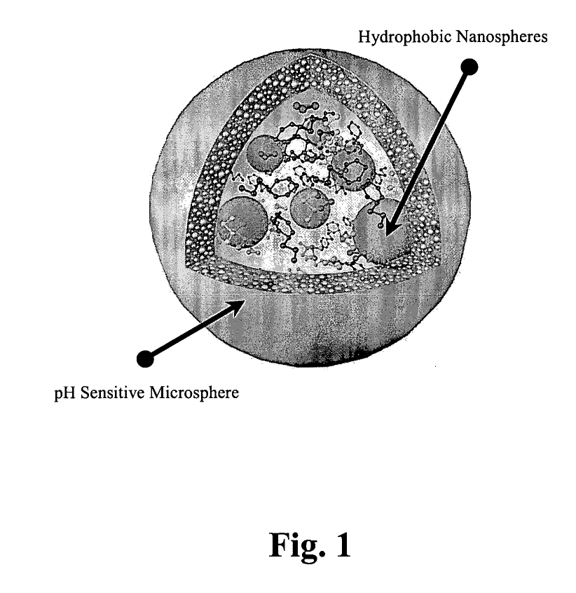

The controlled delivery system of the present invention is a free-flowing powder consisting of solid hydrophobic nanospheres comprising at least one pharmaceutical active ingredient encapsulated in a pH sensitive microsphere, as shown in FIG. 1. The composition is activated by changes in pH in the system proximate environment, to provide targeted delivery, enhanced bioavailability, and prolonged release of pharmaceutical active ingredient(s) over an extended period of time. Pharmaceutical active ingredients can be incorporated in the solid hydrophobic nanospheres, for example, in the pH sensitive microspheres, or in both the nano and microspheres. A first pharmaceutical active ingredient can be incorporated in the nanosphere and a second pharmaceutical active ingredient, which is different from the first pharmaceutical active ingredient, can be incorporated into the microsphere.

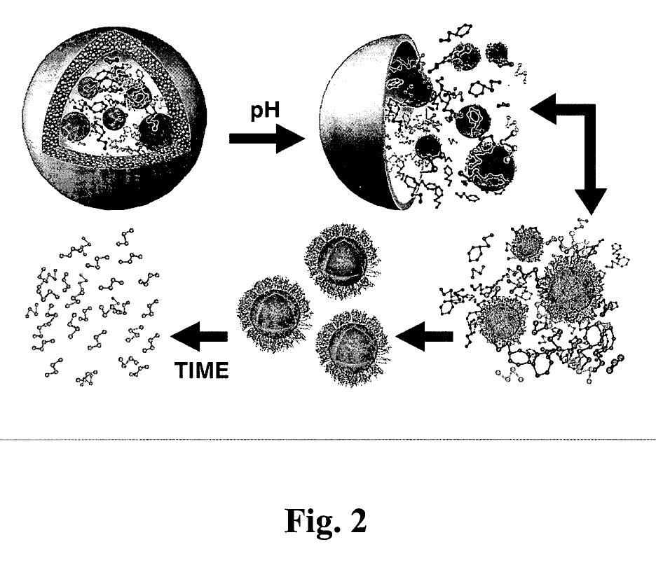

The pharmaceutical active ingredients and the nanospheres are released from the microsphere when the pH of the surrounding environment reaches a desired level or onto certain regions of the gastrointestinal tract including the small intestine (FIG. 2). Upon changes in pH, the microsphere pH sensitive matrix materials dissolve or swell. The dissolution or swelling of the matrix disrupts the microsphere structure and facilitates the release of the nanospheres and the active ingredients.

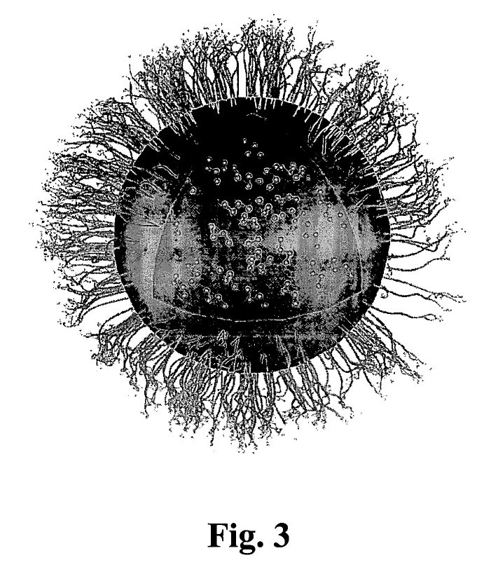

The deposition of the nanospheres onto the target surface is improved by optimizing particle size to ensure entrainment of the nanospheres within a target surface. Various chemical groups and bioadhesive materials can be incorporated in the nanospheres structure, for improving interaction with the target surface (FIG. 3). A cationic surface-active agent creates positively charged nanospheres; an anionic surface-active agent creates negatively charged nanospheres; a nonionic surface active creates neutral charged nanospheres; and a zwitterionic surface-active agent creates variable charged nanospheres. The surface properties of the nanospheres (shown as squiggly lines) can be modified to enhance the affinity of the nanospheres for a particular residue expressed on a cell surface or the affinity of the nanospheres for a cell surface protein or receptor depending on the intended target site.

In one embodiment, the nanospheres of the present invention are bioadhesive. Bioadhesive nanosphere can be created by incorporating a bioadhesive material into the solid hydrophobic matrix of the nanospheres, by incorporating a bioadhesive material in the pH sensitive microsphere matrix, or by using a bioadhesive material in the nanosphere matrix in conjunction with bioadhesive material in the microsphere matrix.

The term “spheres” is intended to describe solid, substantially spherical particulates. It will be appreciated that the term “sphere” includes other particle shapes that can be formed in accordance with the teachings of the present invention.

The term “pH triggered release” is intended to mean that the rate of release is dependent of or regulated by the pH of the system surrounding media or environment.

The pharmaceutical active ingredient encapsulated in the controlled release system of the present invention include, but are not limited to proteins, enzyme, peptides, polypeptide viruses, antigens, (including food allergen), nucleic acids, oligonucleotides, a polynucleotide, plasmid DNA, RNA, proteins, and gene based treatment modalities, including the use of sense, antisense nucleotide sequences, antigens, antibodies, ribozymes, as well as chimeric oligonucleotides constructs for gene correction. These actives may also include DNA or RNA fragments, which code functionally active or inactive or conditionally inactivatable proteins.

The term “anti-sense” is term well-known in the art, i.e., a reverse-orientation nucleic acid that interferes with the function of DNA and/or RNA. This may result in suppression of expression. Natural nucleic acids have a phosphate backbone, artificial nucleic acids may contain other types of backbones and bases. These include PNAs (peptide nucleic acids), phosphothionates, and other variants of the phosphate backbone of native nucleic acids. In addition, DNA and RNA may be single, double, triple, or quadruple stranded.

A nucleic acid can be used to modify genomic or extrachromosomal DNA. This can be achieved by delivering a nucleic acid that is expressed. Alternatively, the nucleic acid can effect a change in the DNA or RNA sequence of the target cell.

The term “gene” refers to a nucleic acid (e.g., DNA) sequence that comprises coding sequences necessary for the production of a polypeptide or precursor (e.g., -myosin heavy chain). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the terms “nucleic acid molecule encoding,” “DNA sequence encoding,” and “DNA encoding,” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

In one embodiment the controlled release system of the present invention can also include a steric stabilizer. A steric stabilizer is a long chain hydrophilic group to stabilize the pharmaceutical active ingredients encapsulated in the controlled release system of the present invention by electrostatic association. Examples include: alkyl groups, PEG chains, polysaccharides, polyethyleneimines, and alkyl amines. Electrostatic interactions are the non-covalent association of two or more substances due to attractive forces between positive and negative charges.

The invention further relates to pharmaceutical formulations that are safe for oral ingestion in various forms, including tablet, capsule, liquid, elixir, powder, granules, etc., comprising the targeted controlled delivery system of the present invention. The targeted controlled delivery system of the present invention can be admixed with food or drinks and it can be self-administered for added convenience rather than requiring the expense of a health professional for administration as is often required for injections and other inoculations.

The multi-component controlled release system of the present invention can comprise from about 0.1% to about 50% by weight hydrophobic matrix, from about 1% to about 50% by weight pH sensitive matrix, from about 0% to about 10% by weight targeting materials, from about 0% to about 20% by weight surface active agents, and from about 0.01% to about 50% by weight active ingredients. The hydrophobic matrix enhances bioavailability and sustains the diffusion rate of the pharmaceutical active ingredients, through the nanospheres and enables them to be released onto the target site over an extended period of time. The microsphere has an average sphere size in the range from about 10 microns to about 100 microns. The nanosphere has an average sphere size in the range from about 0.01 micron to about 1 micron and has a melting point in the range from about 30° C. to about 90° C. This linear dimension for any individual sphere represents the length of the longest straight line joining two points on the surface of the sphere.

Another aspect of the present invention pertains to solid hydrophobic nanospheres encapsulated in a pH sensitive microsphere that can be loaded with pharmaceutical active ingredients for targeted delivery of these actives into the cell and/or its nucleus for gene therapy, nucleic acid vaccination, administration of nucleic acid based drugs or gene based treatment modalities, including the use of sense, antisense nucleotide sequences, antigens, antibodies, ribozymes, as well as chimeric oligonucleotides constructs for gene correction. These actives may also include DNA or RNA fragments, which code functionally active or inactive or conditionally inactivatable proteins.

I. Matrix Materials for Forming the Nanospheres

Considerations in the selection of the matrix material include good barrier properties to the active ingredients, low toxicity and irritancy, stability, integrity, and high loading capacity for the active agents of interest. Suitable wax materials for the compositions and devices of the present invention are inert nontoxic materials with a melting point range between about 25° C. and about 150° C. and penetration point of about 1 to about 10. Examples of wax materials include natural waxes, synthetic waxes and mixtures thereof. Suitable waxes also include natural, regenerated, or synthetic food approved waxes including animal waxes such as beeswax, vegetable waxes such as carnauba, candelilla, sugar cane, rice bran, and bayberry wax, mineral waxes such as petroleum waxes including paraffin and microcrystalline wax, and mixtures thereof.

Other wax materials that are known to those skilled in the art and suitable materials as described in “Industrial Waxes” Vol. I and II, by Bennett F. A. I. C., published by Chemical Publishing Company Inc., 1975 and Martindale, “The Extra Pharmacopoeia,” The Pharmaceutical Press, 28th Edition pp. 1063-1072, 1982 can be used in the present invention.

Suitable fat materials and/or glyceride materials which can be used as matrix materials for forming the nanospheres of the present invention include, but are not limited to, the following classes of lipids: mono-, di and triglycerides, phospholipids, sphingolipids, cholesterol and steroid derivatives, terpenes and vitamins.

The fat material of the present invention can be a glyceride selected from monoglycerides, diglycerides, glyceryl monostearate, glyceryl tristearate and mixtures thereof. Other fat materials which can be used are hydrogenated palm oil, hydrogenated palm kernel oil, hydrogenated peanut oil, hydrogenated rapeseed oil, hydrogenated rice bran oil, hydrogenated soybean oil, hydrogenated cottonseed oil, hydrogenated sunflower oil, partially hydrogenated soybean oil, partially hydrogenated cottonseed oil, and mixtures thereof.

Examples of solid fat materials which can be used in the present invention, include solid hydrogenated castor and vegetable oils, hard fats, and mixtures thereof. Other fat materials which can be used include triglycerides of food grade purity, which can be produced by synthesis or by isolation from natural sources. Natural sources can include animal fat or vegetable oil, such as soy oil, as a source of long chain triglycerides (LCT). Other triglycerides suitable for use in the present invention are composed of a majority of medium length fatty acids (C10-C18), denoted medium chain triglycerides (MCT). The fatty acid moieties of such triglycerides can be unsaturated or polyunsaturated and mixtures of triglycerides having various fatty acid material.

Phospholipids which can be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and beta-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophosphoethanolamine. Synthetic phospholipids with asymmetric acyl chains (e.g., with one acyl chain of 6 carbons and another acyl chain of 12 carbons) can also be used.

Steroids which can be used include as fat materials, but are not limited to, cholesterol, cholesterol sulfate, cholesterol hemisuccinate, 6-(5-cholesterol 3 beta-yloxy) hexyl6-amino-6-deoxy-1-thio-alpha-D-galactopyranoside, 6-(5-cholesten-3 beta-tloxy)hexyl-6-amino-6-deoxyl-1-thio-alpha-D mannopyranoside and cholesteryl)4′-trimethyl 35 ammonio)butanoate. Additional lipid compounds as fat material which can be used include tocopherol and derivatives, and oils and derivatized oils such as stearlyamine.

The fat material can be fatty acids and derivatives thereof which can include, but are not limited to, saturated and unsaturated fatty acids, odd and even number fatty acids, cis and trans isomers, and fatty acid derivatives including alcohols, esters, anhydrides, hydroxy fatty acids and prostaglandins. Saturated and unsaturated fatty acids that can be used include, but are not limited to, molecules that have between 12 carbon atoms and 22 carbon atoms in either linear or branched form. Examples of saturated fatty acids that can be used include, but are not limited to, lauric, myristic, palmitic, and stearic acids. Examples of unsaturated fatty acids that can be used include, but are not limited to, lauric, physeteric, myristoleic, palmitoleic, petroselinic, and oleic acids. Examples of branched fatty acids that can be used include, but are not limited to, isolauric, isomyristic, isopalmitic, and isostearic acids and isoprenoids. Fatty acid derivatives include 12-(((7′-diethylaminocoumarin-3yl)carbonyl)methylamino)-octadecanoic acid; N-[12-(((7′diethylaminocoumarin-3-yl)carbonyl)methyl-amino)octadecanoyl]-2-aminopalmitic acid, N succinyl-dioleoylphosphatidylethanol amine and palmitoyl-homocysteine; and/or combinations thereof. Mono, di and triglycerides or derivatives thereof that can be used include, but are not limited to, molecules that have fatty acids or mixtures of fatty acids between 6 and 24 carbon atoms, digalactosyldiglyceride, 1,2-dioleoyl-sn-glycerol; 1,2-cdipalmitoyl-sn-3 succinylglycerol; and 1,3-dipalmitoyl-2-succinylglycerol.

The nanospheres of the present invention can have a melting point in the range from about 30° C. to about 90° C., preferably from about 40° C. to about 90° C. The melting point of the spheres is typically a function of the carrier matrix employed. Accordingly, preferred matrix materials have a melting point in the range of about 50° C. to about 80° C., preferably from about 60° C. to about 70° C. It should be understood that it is the melting point of the sphere rather than the melting point of the carrier matrix that is important for use of the carrier system of the present invention.

II. Materials for Forming a Microsphere Matrix

The microsphere can be composed of purely pH sensitive materials or of a mixture of pH sensitive materials and water sensitive or bioadhesive materials.

pH Sensitive Materials

Any material and structural form may be used as the pH-sensitive or salt-sensitive trigger means that maintains the integrity of the microsphere until triggered by a solution of the desired pH. Typically, the trigger pH is between about 3 to 12, although in some applications it may be higher or lower. The trigger pH is the threshold pH value or range of values at which either above or below the trigger pH the pH-sensitive material degrades, and/or dissolves. The microsphere can be formed to be stable in solutions and then as the pH rises above the trigger pH the microspheres are activated. Likewise, microspheres can be formed to be stable in solutions and as the pH drops below the trigger pH the microspheres are activated. Once activated, the active ingredients and the nanospheres are released.

In one embodiment a pH-sensitive trigger means is used that the microsphere is capable of becoming more permeable to water and/or losing physical strength following triggering by a solution of the desired pH, either above or below the trigger pH, or salt concentration. In another embodiment a pH-sensitive trigger means is used to hold together two nanosphere portions. The trigger means is capable of losing its adhesive quality or strength, such as to degrade or dissolve, following triggering by a solution of the desired pH, either above or below the trigger pH, or following a change in salt concentration. The reduction in adhesion strength allows the hydrostatic pressure inside the microsphere core to push apart the nanospheres portions held together by the adhesive trigger means, thus releasing the contents.

The pH-sensitive materials can be insoluble solids in acidic or basic aqueous solutions, which dissolve, or degrade and dissolve, as the pH of the solution is neutral. The pH-sensitive materials can be insoluble solids in acidic or basic aqueous solutions which dissolve, or degrade and dissolve, as the pH of the solution rises above or drops below a trigger pH value.

Exemplary pH-sensitive materials include copolymers of acrylate polymers with amino substituents, acrylic acid esters, polyacrylamides, phthalate derivatives (i.e., compounds with covalently attached phthalate moieties) such as acid phthalates of carbohydrates, amylose acetate phthalate, cellulose acetate phthalate, other cellulose ester phthalates, cellulose ether phthalates, hydroxy propyl cellulose phthalate, hydroxypropyl ethylcellulose phthalate, hydroxypropyl methyl cellulose phthalate, methyl cellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinyl acetate phthalate copolymer, styrene and maleic acid copolymers, formalized gelatin, gluten, shellac, salol, keratin, keratin sandarac-tolu, ammoniated shellac, benzophenyl salicylate, cellulose acetate trimellitate, cellulose acetate blended with shellac, hydroxypropylmethyl cellulose acetate succinate, oxidized cellulose, polyacrylic acid derivatives such as acrylic acid and acrylic ester copolymers, methacrylic acid and esters thereof, vinyl acetate and crotonic acid copolymers.

Examples of suitable pH sensitive polymers for use are the Eudragit® polymers series from Rohm America Inc., a wholly-owned subsidiary of Degussa-Huls Corporation, headquartered in Piscataway, N.J., and an affiliate of Rohm GmbH of Darmstadt, Germany. EUDRAGIT® L 30 D-55 and EUDRAGIT® L 100-55, pH dependent anionic polymer that is soluble at pH above 5.5 and insoluble blow pH 5. These polymers can be utilized for targeted drug delivery in the duodenum. EUDRAGIT® L 100 pH dependent anionic polymer that is soluble at pH above 6.0 for targeted drug delivery in the jejunum. EUDRAGIT® S 100 pH dependent anionic polymer that is soluble at pH above 7.0 for targeted drug delivery in the ileum. EUDRAGIT® E 100 and EUDRAGIT® EPO, pH dependent cationic polymer, soluble up to pH 5.0 and insoluble above pH 5.0.

Dependent cationic polymer, soluble up to pH 5.0 and insoluble above pH 5.0. Accordingly, suitable pH sensitive materials degrade or dissolve when said pH sensitive microsphere contacts a solution having a pH greater than about 5.

Additional pH-sensitive materials include poly functional polymers containing multiple groups that become ionized as the pH drops below their pKa. A sufficient quantity of these ionizable groups must be incorporated in the polymer such that in aqueous solutions having a pH below the pKa of the ionizable groups, the polymer dissolves. These ionizable groups can be incorporated into polymers as block copolymers, or can be pendent groups attached to a polymer backbone, or can be a portion of a material used to crosslink or connect polymer chains. Examples of such ionizable groups include polyphosphene, vinyl pyridine, vinyl aniline, polylysine, polyornithine, other proteins, and polymers with substituents containing amino moieties.

pH-sensitive polymers which are relatively insoluble and impermeable at the pH of the stomach, but which are more soluble and permeable at the pH of the small intestine and colon include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

Other pH-sensitive polymers include shellac; phthalate derivatives, particularly cellulose acetate phthalate, polyvinylacetate phthalate, and hydroxypropylmethylcellulose phthalate; polyacrylic acid derivatives, particularly polymethyl methacrylate blended with acrylic acid and acrylic ester copolymers; and vinyl acetate and crotonic acid copolymers.

Anionic acrylic copolymers of methacrylic acid and methylmethacrylate are also particularly useful coating materials for delaying the release of compositions and devices until the compositions and devices have moved to a position in the small intestine which is distal to the duodenum. Copolymers of this type are available from RohmPharma Corp, under the trade names Eudragit-L.R™ and Eudragit-S.R™, are anionic copolymers of methacrylic acid and methylmethacrylate. The ratio of free carboxyl groups to the esters is approximately 1:1 in Eudragit-L.R™ and approximately 1:2 in Eudragit-S.R™. Mixtures of Eudragit-L.R™ and Eudragit-S.R™ can also be used.

Other pH-sensitive materials are cationic pH sensitive polymers and copolymers that are water insoluble at pH 9 and above and are water soluble or water dispersible at pH 7.

The pH-sensitive and salt sensitive materials can be blended with an inert water sensitive material. By inert is meant a material that is not substantially affected by a change in pH or salt concentration in the triggering range. By altering the proportion of a pH-sensitive material to inert material the time lag subsequent to triggering and prior to release can be tailored.

In an embodiment of the present invention, the micro sphere is formed of a pH sensitive material which is substantially insoluble and impermeable at the pH of the stomach, and is more soluble and permeable at the pH of the small intestine. Preferably, the micro spheres are substantially insoluble and impermeable at pH less than about 5.0, and water-soluble at pH greater than about 5.0. pH-sensitive polymers which are relatively insoluble and impermeable at the pH of the stomach, but which are more soluble and permeable at the pH of the small intestine and colon include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

Preferred pH-sensitive polymers include shellac; phthalate derivatives, particularly cellulose acetate phthalate, polyvinylacetate phthalate, and hydroxypropylmethylcellulose phthalate; polyacrylic acid derivatives, particularly polymethyl methacrylate blended with acrylic acid and acrylic ester copolymers; vinyl acetate; crotonic acid copolymers and Eudragit® polymers series from Rohm America Inc.

Water Sensitive Materials

Water-sensitive materials can be mixed with the pH or salt sensitive materials to form the microspheres of the present invention. Suitable water sensitive materials comprise polyvinyl pyrrolidone, water soluble celluloses, polyvinyl alcohol, ethylene maleic anhydride copolymer, methyl vinyl ether maleic anhydride copolymer, polyethylene oxides, water soluble polyamide or polyester, copolymers or homopolymers of acrylic acid such as polyacrylic acid, polystyrene acrylic acid copolymers or starch derivatives, polyvinyl alcohol, polysaccharides, hydrocolloids, natural gums, proteins, and mixtures thereof. Examples of synthetic water sensitive polymers which are useful for the invention include polyvinyl pyrrolidone, water soluble celluloses, polyvinyl alcohol, ethylene maleic anhydride copolymer, methylvinyl ether maleic anhydride copolymer, acrylic acid copolymers, anionic polymers of methacrylic acid and methacrylate, cationic polymers with dimethyl-aminoethyl ammonium functional groups, polyethylene oxides, water soluble polyamide or polyester.

Examples of water soluble hydroxyalkyl and carboxyalkyl celluloses include hydroxyethyl and carboxymethyl cellulose, hydroxyethyl and carboxyethyl cellulose, hydroxymethyl and carboxymethyl cellulose, hydroxypropyl carboxymethyl cellulose, hydroxypropyl methyl carboxyethyl cellulose, hydroxypropyl carboxypropyl cellulose, hydroxybutyl carboxymethyl cellulose, and the like. Also useful are alkali metal salts of these carboxyalkyl celluloses, particularly and preferably the sodium and potassium derivatives.

The polyvinyl alcohol useful in the practice of the invention is partially and fully hydrolyzed polyvinyl acetate, termed “polyvinyl alcohol” with polyvinyl acetate as hydrolyzed to an extent, also termed degree of hydrolysis, of from about 75% up to about 99%. Such materials are prepared by means of any of Examples I-XIV of U.S. Pat. No. 5,051,222 issued on Sep. 24, 1991, the specification for which is incorporated by reference herein.

Polyvinyl alcohol useful for practice of the present invention is Mowiol® 3-83, having a molecular weight of about 14,000 Da and degree of hydrolysis of about 83%, Mowiol® 3-98 and a fully hydrolyzed (98%) polyvinyl alcohol having a molecular weight of 16,000 Da commercially available from Gehring-Montgomery, Inc. of Warminster Pa. Other suitable polyvinyl alcohols are: AIRVOL® 205, having a molecular weight of about 15,000-27,000 Da and degree of hydrolysis of about 88%, and VINEX® 1025, having molecular weight of 15,000-27,000 Da degree of hydrolysis of about 99% and commercially available from Air Products & Chemicals, Inc. of Allentown, Pa.; ELVANOL® 51-05, having a molecular weight of about 22,000-26,000 Da and degree of hydrolysis of about 89% and commercially available from the DuPont Company, Polymer Products Department, Wilmington, Del.; ALCOTEX® 78 having a degree of hydrolysis of about 76% to about 79%, ALCOTEX® F88/4 having a degree of hydrolysis of about 86% to about 88% and commercially available from the Harlow Chemical Co. Ltd. of Templefields, Harlow, Essex, England CM20 2BH; and GOHSENOL® GL-03 and GOHSENOL® KA-20 commercially available from Nippon Gohsei K.K., The Nippon Synthetic Chemical Industry Co., Ltd., of No. 9-6, Nozaki Cho, Kita-Ku, Osaka, 530 Japan.

Suitable polysaccharides are polysaccharides of the non-sweet, coloidally-soluble types, such as natural gums, for example, gum arabic, starch derivates, dextrinized and hydrolyzed starches, and the like. A suitable polysaccharide is a water dispersible, modified starch commercially available as Capule®, N-Lok®, Hi-Cap™ 100 or Hi-Cap™ 200 commercially available from the National Starch and Chemical Company of Bridgewater, N.J.; Pure-Cote™, commercially available from the Grain Processing Corporation of Muscatine, Iowa. In the preferred embodiment the natural gum is a gum arabic, commercially available from TIC Gums Inc., Belcamp, Midland. Suitable hydrocolloids are xanthan, maltodextrin, galactomanan or tragacanth, preferably maltodextrins such as Maltrin™ M100, and Maltrin™ M150, commercially available from the Grain Processing Corporation of Muscatine, Iowa.

Bioadhesive Materials

An orally ingested drug delivery system can adhere to either the epithelial surface or the mucus. For the delivery of bioactive active ingredients, it is advantageous to have the system adhere to the epithelium rather than solely to the mucous layer, although mucoadhesion can also substantially improve bioavailability. For some types of imaging purposes, adhesion to both the epithelium and mucus is desirable whereas in pathological states, such as in the case of gastric ulcers or ulcerative colitis, adhesion to cells below the mucous layer may occur. Duchene, et al., Drug Dev. Ind. Pharm. 14(2&3), 283-318 (1988), reviews the pharmaceutical and medical aspects of bioadhesive systems for drug delivery. “Bioadhesion” is defined as the ability of a material to adhere to a biological tissue for an extended period of time. Bioadhesion is a solution to the problem of inadequate residence time resulting from the stomach emptying and intestinal peristalsis, and from displacement by ciliary movement. For sufficient bioadhesion to occur, an intimate contact is needed between the bioadhesive and the receptor tissue, the bioadhesive must penetrate into the crevice of the tissue surface and/or mucus, and mechanical, electrostatic, or chemical bonds form. Bioadhesive properties of the polymers are affected by both the nature of the polymer and by the nature of the surrounding media.

Incorporating bioadhesive polymers in the microsphere of the present invention can be utilized to control or increase the absorption of the nanosphere through the mucosal lining, or to further delay transit of the nanosphere through the gastrointestinal passages.

A bioadhesive polymer as used in the disclosure is one that binds to mucosal epithelium under normal physiological conditions. Bioadhesion in the gastrointestinal tract proceeds in two stages: (1) viscoelastic deformation at the point of contact of the synthetic material into the mucus substrate, and (2) formation of bonds between the adhesive synthetic material and the mucus or the epithelial cells. In general, adhesion of polymers to tissues can be achieved by (i) physical or mechanical bonds, (ii) primary or covalent chemical bonds, and/or (iii) secondary chemical bonds such as ionic. Physical or mechanical bonds can result from deposition and inclusion of the bioadhesive material in the crevices of the mucus or the folds of the mucosa. Secondary chemical bonds, contributing to bioadhesive properties, can comprise dispersive interactions such as Van der Waals interactions and stronger specific interactions, such as hydrogen bonds. Hydrophilic functional groups primarily responsible for forming hydrogen bonds include hydroxyl and the carboxylic groups. Suitable bioadhesive polymers for use in the present invention include bioerodible hydrogels as described by H. S. Sawhney, C. P. Pathak and J. A. Hubell in Macromolecules. 1993, 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butyl methacrylate), poly(isobutyl methacrylate), poly(hexl methacrylate), poly(isodecl methacrylate), poly(lauryl methacrylate), poly(phenyl methacrylate), poly(methyl acrylate), poly(isopropyl acrylate), poly(isobutyl acrylate), and poly(octadecl acrylate) and poly(fumaric-co-sebacic)acid.

Polymers with enhanced bioadhesive properties can be provided wherein anhydride monomers or oligomers are incorporated into the polymer. The oligomer excipients can be blended or incorporated into a wide range of hydrophilic and hydrophobic polymers including proteins, polysaccharides and synthetic biocompatible polymers. Anhydride oligomers can be combined with metal oxide particles to improve bioadhesion in addition to the use of organic additives alone. Organic dyes because of their electronic charge and hydrophobicity/hydrophilicity can either increase or decrease the bioadhesive properties of polymers when incorporated into the polymers. The incorporation of oligomer compounds into a wide range of different polymers which are not normally bioadhesive can be used to increase the adherence of the polymer to tissue surfaces such as mucosal membranes.

III. Targeting Mechanism

Targeting ligands according to the present invention are any molecules which bind to specific types of cells in the body. These may be any type of molecule for which a cellular receptor exists. Preferably the cellular receptors are expressed on specific cell types only. Examples of targeting ligands which may be used are hormones, antibodies, cell-adhesion molecules, saccharides, drugs, and neurotransmitters.

The nanospheres can be targeted specifically or non-specifically through the selection of the pH of the material forming the microsphere, the size of the nanosphere, and/or incorporation or attachment of a ligand to the nanospheres. For example, biologically active molecules, or molecules affecting the charge, lipophilicity or hydrophilicity of the nanospheres, can be attached to the surface of the nanospheres. Additionally, molecules can be attached to the nanospheres which minimize tissue adhesion, or which facilitate specific targeting of the nanosphere in vivo. Representative targeting molecules include antibodies, lectins, and other molecules which are specifically bound by receptors on the surfaces of cells of a particular type.

The term “cell recognition component,” as used herein, refers to a molecule capable of recognizing a component on a surface of a targeted cell. Cell recognition components may include an antibody to a cell surface antigen, a ligand for a cell surface receptor, such as cell surface receptors involved in receptor-mediated endocytosis, peptide hormones, and the like. In one embodiment of the present invention, the nanospheres are modified with lectins attached to the nanosphere surface and targeted to mucosal epithelium of the small intestine and are absorbed into the systemic circulation and lymphatic circulation. In an embodiment of the present invention, carbohydrates or lectins are used to target the nanospheres of the present invention to M cells and Peyer's Patch cells of the small intestine. In another embodiment of the present invention, lectins which bind to fucosyl sugars are used to modify the nanospheres. Lectins are a heterogenous group of proteins or glycoproteins that recognize carbohydrate residues on cell surface glycoconjugates with a high degree of specificity. Examples of lectins that can be used to modify the nanospheres of the present invention, include but are not limited to, lectins specific for binding to fucosyl glycoconjugates, such as Ulex Europeas Agglutinin I (UEA); lectins specific for binding to galactose/N-acetylgalactoseamine, such as Phaseolus vulgaris haemagglutinin (PHA), tomato lectin (Lycopersicon esculentum) (TL), wheat germ agglutinin (WGA); lectins specific for binding to mannose, such as, Galanthus nivalis agglutinin (GNA); lectins specific for mannose and/or glucose, such as, con A/concavalan A. (See e.g., Lehr et al., 1995, in Lectins Biomedical Perspectives, pp. 117-140, incorporated by reference into this application). The targeting molecules can be derivatized if desired. See e.g., Chen et al., 1995, Proceed. Internat. Symp. Control. Rel. Bioact. Mater. 22 and Cohen, WO 9503035, incorporated by reference into this application.

In another embodiment of the invention, the nanospheres of the present invention can be modified with viral proteins or bacterial proteins that have an affinity for a particular residue expressed on a cell surface or that have an affinity for a cell surface protein or receptor. Examples of such proteins include, but are not limited to, cholera toxin B subunit, and bacterial adhesotopes.

In yet another embodiment of the present invention, the nanospheres of the present invention can be modified with monoclonal antibodies or fragments of antibodies which target the nanospheres to a particular cell type. The nanospheres of the present invention can be modified with ligands for specific mucosal cell surface receptors and proteins. As used herein, the term “ligand” refers to a ligand attached to a nanosphere which adheres to the mucosa in the intestine or can be used to target the nanospheres to a specific cell type in the G-I tract or following absorption of the nanospheres onto the mucosa in the intestine. Suitable ligands can include ligands for specific cell surface proteins and antibodies or antibody fragments immunoreactive with specific surface molecules. Suitable ligands can also include less specific targeting ligands such as coatings of materials which are bioadhesive, for example alginate and polyacrylate.

IV. Active Ingredients

The pharmaceutical active ingredient encapsulated in the controlled release system of the present invention include, but are not limited to proteins, peptides, viruses, antigens, nucleic acids, oligonucleotides, a polynucleotide, plasmid DNA, RNA, proteins, and gene based treatment modalities, including the use of sense, antisense nucleotide sequences, antigens, antibodies, ribozymes, as well as chimeric oligonucleotides constructs for gene correction. These actives may also include DNA or RNA fragments, which code functionally active or inactive or conditionally inactivatable proteins.

V. Processing Method

Va. Nanospheres

The encapsulated active agent in the nanospheres of the present invention can be prepared by the steps of (1) heating hydrophobic materials to a temperature above the melting point to form a melt, (2) dissolving or dispersing the active agent in the melt, (3) emulsifying the melt in the aqueous phase; and (4) cooling the dispersion to ambient temperature to form a fine suspension.

The active ingredients can be incorporated into hydrophobic solid nanospheres, the pH sensitive microsphere, or in both the nano and micro spheres.

Vb. Microspheres The controlled release system of the present invention can be prepared by the steps of (a) incorporating the selected active agents into the hydrophobic interior of the nanospheres, (b) forming an aqueous mixture comprising one or more active agents, the nanospheres, and a pH sensitive material, and (c) spray drying the mixture of the present invention to form a dry powder composition. Accordingly, the nanospheres can be encapsulated into the microsphere structure. One or more of the active agents which can be the same or different than the active agents incorporated in the nanosphere can be incorporated into the microsphere structure.

A process for producing the multi component controlled release system includes the following steps:

(i) heating a hydrophobic material to a temperature above the melting point to form a melt;

(ii) dissolving or dispersing the selected first active agent into the melt;

(iii) dissolving or dispersing a second active agent, and the pH sensitive materials, in the aqueous phase and heating it to above the melting temperature of the hydrophobic material;

(iv) mixing the hot melt with the aqueous phase to form a dispersion;

(v) high shear homogenization of the dispersion at a temperature above the melting temperature until a homogeneous fine dispersion is obtained having a sphere size of from about 1 microns to about 2 microns;

(vi) cooling the dispersion to ambient temperature; and

(vii) spray drying the emulsified mixed suspension to form a dry powder composition.

Homogenization can be accomplished in any suitable fashion with a variety of mixers known in the art such as simple paddle or ribbon mixers although other mixers, such as ribbon or plow blenders, drum agglomerators, and high shear mixers may be used. Suitable equipment for this process include a model Rannie 100 lab homogenizer available from APV Gaulin Inc., Everett, Mass., a rotor stator high shear mixer available from Silverson Machines, of East Long Meadow, Mass., or Scott Processing Equipment Corp. of Sparta, N.J., and other high sear mixers.

The suspension is spray dried to remove the excess water. Spray drying is well known in the art and been used commercially in many applications, including foods where the core material is a flavoring oil and cosmetics where the core material is a fragrance oil. Cf. Balassa, “Microencapsulation in the Food Industry,” CRC Critical Review Journal in Food Technology, July 1971, pp 245-265; Barreto, “Spray Dried Perfumes for Specialties, Soap and Chemical Specialties,” December 1966; Maleeny, Spray Dried Perfumes, Soap and San Chem, January 1958, pp. 135 et seq.; Flinn and Nack, “Advances in Microencapsulation Techniques,” Batelle Technical Review, Vol. 16, No. 2, pp. 2-8 (1967); U.S. Pat. Nos. 5,525,367; and 5,417,153 which are incorporated herein as references.

The use of pH activated microspheres which provide varying rates of diffusion are contemplated. For example, the active ingredients encapsulated in the pH activated microspheres may diffuse at any of the rates of the following:

(i) at steady-state or zero-order release rate in which there is a substantially continuous release per unit of time;

(ii) a first-order release rate in which the rate of release declines towards zero with time; and

(iii) a delayed release in which the initial rate is slow, but then increases with time.

Nanospheres formed of a hydrophobic material provide a controlled release system in order to release the active agent over an extended period of time by molecular diffusion. Active agents in the hydrophobic matrix of the nanospheres can be released by transient diffusion. The theoretical early and late time approximation of the release rate of the active ingredients dissolved in the hydrophobic matrix of the nanospheres can be calculated from the following equations:

Early time approximation

$$\begin{matrix}( {m}_{t} / {m}_{s e c} ) < 0.4 \\ \frac{{M}_{t}}{{M}_{∞}} = 4 {( \frac{{D}_{p} t}{Π {r}^{2}} )}^{1 / 2} - \frac{{D}_{p} t}{{r}^{2}} & ( 1 ) \\ \frac{ⅆ {M}_{t} / {M}_{∞}}{ⅆ t} = 2 {( \frac{{D}_{p}}{Π {r}^{2} t} )}^{1 / 2} - \frac{{D}_{p}}{{r}^{2}} & ( 2 )\end{matrix}$$

Late time approximation

$$\begin{matrix}( {m}_{t} / {m}_{∞} ) > 0.6 \\ \frac{{M}_{t}}{{M}_{∞}} = 1 - \frac{4}{{( 2.405 )}^{2}} exp ( \frac{- {( 2.405 )}^{2} {D}_{p} t}{{r}^{2}} ) & ( 3 ) \\ \frac{ⅆ {M}_{t} / {M}_{∞}}{ⅆ t} = 1 - \frac{4 {D}_{p}}{{r}^{2}} exp ( \frac{- {( 2.405 )}^{2} {D}_{p} t}{{r}^{2}} ) & ( 4 )\end{matrix}$$

wherein:

r is the radius of the cylinder,

m∞ is the amount fragrance released from the controlled release system after infinite time;

mₜ is the amount fragrance released from the controlled release system after time t; and

Dₚ is the diffusion coefficient of the fragrance or aroma chemical in the matrix.

The release rate for releasing the active agents from the hydrophobic nanospheres is typically slower than the release rate for releasing active agent from the pH sensitive matrix. The active agents can be selected to be incorporated into either the hydrophobic nanospheres or the pH sensitive matrix depending on the desired time for release of the active agents. For example, the pH sensitive matrix formed in accordance with the present invention can release the first active agent at a predetermined pH to provide a “burst” with continued release of the first active agent and nanospheres formed in accordance with the present invention can release the active agent depending on the release rate from an initial time such as a day or within few days, up to a period of few weeks.

In the preferred embodiment, the active agent is present at a level from about 0.01% to about 60%, preferably from about 1% to about 50% by weight of the microsphere. In the preferred embodiment, the nanospheres are generally present in the pH sensitive matrix at a level from about 1% to about 80%, preferably from about 1% to about 60% by weight of the matrix material with the balance being the active agents, and the pH sensitive materials. In the preferred embodiment, the pH sensitive matrix is generally present at a level from about 1% to about 80%, preferably from about 1% to about 60% by weight of the matrix material with the balance being the active agents, and the hydrophobic materials.

The compositions preferably are formulated in unit dosage form, meaning physically discrete units suitable as a unitary dosage, or a predetermined fraction of a unitary dose to be administered in a single or multiple dosage regimen to human subjects and other mammals, each unit containing a predetermined quantity of active material calculated to produce the desired therapeutic effect in association with a suitable pharmaceutical excipient.

The invention can be further illustrated by the following examples thereof, although it will be understood that these examples are included merely for purposes of illustration and are not intended to limit the scope of the invention unless otherwise specifically indicated. All percentages, ratios, and parts herein, in the Specification, Examples, and claims, are by weight and are approximations unless otherwise stated.

### Preparation of a pH Sensitive Multi Component Controlled Delivery Systems

### EXAMPLE 1

The following procedure is used for the preparation of multi component controlled release system with a drug as the active agent in the hydrophobic nanosphere matrix. The nanosphere hydrophobic matrix is candelilla wax, commercially available from Strahl & Pitsch Inc. of West Babylon, N.Y. Bovine serum albumin (BSA) (commercially available from Sigma-Aldrich Co., St. Louis, Mont.) was used as a model protein. The microsphere pH sensitive matrix is EUDRAGIT® S 100 pH dependent anionic polymer solubilizing above pH 7.0 for delivery of the drug in the small intestine and colon (commercially available from Rohm America Inc. of Piscataway, N.J.) for releasing the nanospheres in the duodenum.

25 grams of candelilla wax and 20 grams of castor oil are placed in an oven at 60° C. and allowed to melt. 500 grams of deionized water are placed into 1 gallon vessel, fitted with an all-purpose silicon rubber heater (Cole-Palmer Instrument Company). 50 grams of Eudragit® S 100 (commercially available from Rohm America Inc. of Piscataway, N.J.) were added to the water and the aqueous solution is heated to 70° C. while mixing it with a propeller mixer. The candelilla wax is removed from the oven. 5 grams of bovine serum albumin are mixed into the melt by hand with a glass rod. The protein/wax mixture is poured into the aqueous solution and the dispersion is homogenized at 20,000 psi using a Rannie 100 lab homogenizer available from APV Gaulin Inc. The dispersion is cooled to ambient temperature by passing it through a tube-in-tube heat exchanger (Model 00413, Exergy Inc., Hanson, Mass.) to form a suspension. The resulting suspension is spray dried with a Bowen Lab Model Drier (at Spray-Tek of Middlesex, N.J.) utilizing 250 c.f.m of air with an inlet temperature of 280° F., and outlet temperature of 225° F. and a wheel speed of 45,000 r.p.m to produce a free flowing, dry powder, consisting of 5% bovine serum albumin encapsulated in the solid hydrophobic nanospheres.

### EXAMPLE 2

The following procedure is used for the preparation of multi component controlled release system with a drug as the active agent in the hydrophobic nanosphere matrix. The nanosphere hydrophobic matrix is beeswax and castor oil, commercially available from Strahl & Pitsch Inc. of West Babylon, N.Y. Bovine serum albumin (BSA) (commercially available from Sigma-Aldrich Co., St. Louis, Mont.) was used as a model protein. The microsphere pH sensitive matrix is EUDRAGIT® S 100 pH dependent anionic polymer solubilizing above pH 7.0 for delivery of the drug in the small intestine and colon (commercially available from Rohm America Inc. of Piscataway, N.J.) for releasing the nanospheres in the duodenum, the water sensitive matrix is Hi-Cap™ 100 (commercially available from the National Starch and Chemical Company of Bridgewater, N.J.).

20 grams of beeswax wax and 25 grams of castor oil are placed in an oven at 60° C. and allowed to melt. 500 grams of deionized water are placed into 1 gallon vessel, fitted with an all-purpose silicon rubber heater (Cole-Palmer Instrument Company). 40 grams of Eudragit® S 100 (commercially available from Rohm America Inc. of Piscataway, N.J.) and 10 grams of Hi-Cap™ 100 (commercially available from the National Starch and Chemical Company of Bridgewater, N.J.) were added to the water and the aqueous solution is heated to 70° C. while mixing it with a propeller mixer. The melt is removed from the oven and 5 grams of bovine serum albumin are mixed into the melt by hand with a glass rod. The protein/wax mixture is poured into the aqueous solution and the dispersion is homogenized at 20,000 psi using a Rannie 100 lab homogenizer available from APV Gaulin Inc. The dispersion is cooled to ambient temperature by passing it through a tube-in-tube heat exchanger (Model 00413, Exergy Inc., Hanson, Mass.) to form a suspension. The resulting suspension is spray dried with a Bowen Lab Model Drier (at Spray-Tek of Middlesex, N.J.) utilizing 250 c.f.m of air with an inlet temperature of 300° F., and outlet temperature of 225° F. and a wheel speed of 45,000 r.p.m to produce a free flowing, dry powder, consisting of 5% Bovine serum albumin encapsulated in the solid hydrophobic nanospheres.

### EXAMPLE 3

The following procedure is used for the preparation of multi component controlled release system with a drug as the active agent in the hydrophobic nanosphere matrix. The nanosphere hydrophobic matrix is candelilla wax, commercially available from Strahl & Pitsch Inc. of West Babylon, N.Y. The therapeutic enzyme Asparaginase-polyethylene glycol from Escherichia coli (commercially available from Sigma-Aldrich Co., St. Louis, Mont.) was used as a model enzyme. The microsphere pH sensitive matrix is EUDRAGIT® S 100 pH dependent anionic polymer solubilizing above pH 7.0 for delivery of the drug in the small intestine and colon (commercially available from Rohm America Inc. of Piscataway, N.J.) for releasing the nanospheres in the duodenum.

25 grams of candelilla wax and 24 grams of castor oil are placed in an oven at 60° C. and allowed to melt. 500 grams of deionized water are placed into 1 gallon vessel, fitted with an all-purpose silicon rubber heater (Cole-Palmer Instrument Company). 50 grams of Eudragit® S 100 (commercially available from Rohm America Inc. of Piscataway, N.J.) were added to the water and the aqueous solution is heated to 70° C. while mixing it with a propeller mixer. The candelilla wax is removed from the oven. 1 gram of Asparaginase-polyethylene glycol is mixed into the melt by hand with a glass rod. The protein/wax mixture is poured into the aqueous solution and the dispersion is homogenized at 20,000 psi using a Rannie 100 lab homogenizer available from APV Gaulin Inc. The dispersion is cooled to ambient temperature by passing it through a tube-in-tube heat exchanger (Model 00413, Exergy Inc., Hanson, Mass.) to form a suspension. The resulting suspension is spray dried with a Bowen Lab Model Drier (at Spray-Tek of Middlesex, N.J.) utilizing 250 c.f.m of air with an inlet temperature of 280° F., and outlet temperature of 225° F. and a wheel speed of 45,000 r.p.m to produce a free flowing, dry powder, consisting of 1% bovine Asparaginase-polyethylene glycol encapsulated in the solid hydrophobic nanospheres.

### EXAMPLE 4

The following procedure is used for the preparation of multi component controlled release system with a drug as the active agent in the hydrophobic nanosphere matrix. The nanosphere hydrophobic matrix is beeswax and castor oil, commercially available from Strahl & Pitsch Inc. of West Babylon, N.Y. Deoxyribonucleic acid sodium salt from calf thymus (commercially available from Sigma-Aldrich Co., St. Louis, Mont.) was used as a model active ingredient. The microsphere pH sensitive matrix is EUDRAGIT® S 100 pH dependent anionic polymer solubilizing above pH 7.0 for delivery of the drug in the small intestine and colon (commercially available from Rohm America Inc. of Piscataway, N.J.) for releasing the nanospheres in the duodenum, the water sensitive matrix is Hi-Cap™ 100 (commercially available from the National Starch and Chemical Company of Bridgewater, N.J.).

20 grams of beeswax wax and 29 grams of castor oil are placed in an oven at 60° C. and allowed to melt. 500 grams of deionized water are placed into 1 gallon vessel, fitted with an all-purpose silicon rubber heater (Cole-Palmer Instrument Company). 40 grams of Eudragit® S 100 (commercially available from Rohm America Inc. of Piscataway, N.J.) and 10 grams of Hi-Cap™ 100 (commercially available from the National Starch and Chemical Company of Bridgewater, N.J.) were added to the water and the aqueous solution is heated to 70° C. while mixing it with a propeller mixer. The melt is removed from the oven and 1 gram of Deoxyribonucleic acid sodium salt from calf thymus is mixed into the melt by hand with a glass rod. The protein/wax mixture is poured into the aqueous solution and the dispersion is homogenized at 20,000 psi using a Rannie 100 lab homogenizer available from APV Gaulin Inc. The dispersion is cooled to ambient temperature by passing it through a tube-in-tube heat exchanger (Model 00413, Exergy Inc., Hanson, Mass.) to form a suspension. The resulting suspension is spray dried with a Bowen Lab Model Drier (at Spray-Tek of Middlesex, N.J.) utilizing 250 c.f.m of air with an inlet temperature of 300° F., and outlet temperature of 225° F. and a wheel speed of 45,000 r.p.m to produce a free flowing, dry powder, consisting of 1% Deoxyribonucleic acid sodium salt from calf thymus encapsulated in the solid hydrophobic nanospheres.

### EXAMPLE 5

The following procedure is used for the preparation of multi component controlled release system with a drug as the active agent in the hydrophobic nanosphere matrix. The nanosphere hydrophobic matrix is candelilla wax, commercially available from Strahl & Pitsch Inc. of West Babylon, N.Y. The therapeutic enzyme Asparaginase-polyethylene glycol from Escherichia coli (commercially available from Sigma-Aldrich Co., St. Louis, Mont.) was used as a model enzyme. Polyethyleneimine (commercially available from BASF Corporation as Lupasol® FG) was used as a stabilizer. The microsphere pH sensitive matrix is EUDRAGIT® S 100 pH dependent anionic polymer solubilizing above pH 7.0 for delivery of the drug in the small intestine and colon (commercially available from Rohm America Inc. of Piscataway, N.J.) for releasing the nanospheres in the duodenum.

24.5 grams of candelilla wax and 24 grams of castor oil are placed in an oven at 60° C. and allowed to melt. 500 grams of deionized water are placed into 1 gallon vessel, fitted with an all-purpose silicon rubber heater (Cole-Palmer Instrument Company). 50 grams of Eudragit® S 100 (commercially available from Rohm America Inc. of Piscataway, N.J.) were added to the water and the aqueous solution is heated to 70° C. while mixing it with a propeller mixer. The candelilla wax is removed from the oven. 1 grams of Asparaginase-polyethylene glycol and 0.5 grams of polyethyleneimine are mixed and incorporated into the melt by hand with a glass rod. The protein/wax mixture is poured into the aqueous solution and the dispersion is homogenized at 20,000 psi using a Rannie 100 lab homogenizer available from APV Gaulin Inc. The dispersion is cooled to ambient temperature by passing it through a tube-in-tube heat exchanger (Model 00413, Exergy Inc., Hanson, Mass.) to form a suspension. The resulting suspension is spray dried with a Bowen Lab Model Drier (at Spray-Tek of Middlesex, N.J.) utilizing 250 c.f.m of air with an inlet temperature of 280° F., and outlet temperature of 225° F. and a wheel speed of 45,000 r.p.m to produce a free flowing, dry powder, consisting of 1% bovine Asparaginase-polyethylene glycol encapsulated in the solid hydrophobic nanospheres.

### EXAMPLE 6

The following procedure is used for the preparation of multi component controlled release system with a drug as the active agent in the hydrophobic nanosphere matrix. The nanosphere hydrophobic matrix is beeswax and castor oil, commercially available from Strahl & Pitsch Inc. of West Babylon, N.Y. Ribonuclease A from bovine pancreas (RNase A) (commercially available from Sigma-Aldrich Co., St. Louis, Mont.) was used as a model active ingredient. The microsphere pH sensitive matrix is EUDRAGIT® S 100 pH dependent anionic polymer solubilizing above pH 7.0 for delivery of the drug in the small intestine and colon (commercially available from Rohm America Inc. of Piscataway, N.J.) for releasing the nanospheres in the duodenum, the water sensitive matrix is Hi-Cap™ 100 (commercially available from the National Starch and Chemical Company of Bridgewater, N.J.).

20 grams of beeswax wax and 29 grams of castor oil are placed in an oven at 60° C. and allowed to melt. 500 grams of deionized water are placed into 1 gallon vessel, fitted with an all-purpose silicon rubber heater (Cole-Palmer Instrument Company). 40 grams of Eudragit® S 100 (commercially available from Rohm America Inc. of Piscataway, N.J.) and 10 grams of Hi-Cap™ 100 (commercially available from the National Starch and Chemical Company of Bridgewater, N.J.) were added to the water and the aqueous solution is heated to 70° C. while mixing it with a propeller mixer. The melt is removed from the oven and 1 grams of Ribonuclease A from bovine pancreas (RNase A) is mixed into the melt by hand with a glass rod. The protein/wax mixture is poured into the aqueous solution and the dispersion is homogenized at 20,000 psi using a Rannie 100 lab homogenizer available from APV Gaulin Inc. The dispersion is cooled to ambient temperature by passing it through a tube-in-tube heat exchanger (Model 00413, Exergy Inc., Hanson, Mass.) to form a suspension. The resulting suspension is spray dried with a Bowen Lab Model Drier (at Spray-Tek of Middlesex, N.J.) utilizing 250 c.f.m of air with an inlet temperature of 300° F., and outlet temperature of 225° F. and a wheel speed of 45,000 r.p.m to produce a free flowing, dry powder, consisting of 1% Ribonuclease A from bovine pancreas (RNase A) encapsulated in the solid hydrophobic nanospheres.

It is to be understood that the above-described embodiments are illustrative of only a few of the many possible specific embodiments which can represent applications of the principles of the invention. Numerous and varied other arrangements can be readily devised in accordance with these principles by those skilled in the art without departing from the spirit and scope of the invention.

What is claimed is:

1. A controlled release composition comprising:
a plurality of solid nano-spheres, each of said solid nano-spheres, said plurality of solid nano-spheres being encapsulated in a pH sensitive or salt sensitive micro-sphere, said pH sensitive or salt sensitive micro-sphere is formed of a pH sensitive or salt sensitive matrix material, and a first active agent incorporated into said solid nano-spheres or said microsphere or in both said solid nano-sphere and said micro-sphere, wherein said first active agent is a nucleic acid.

2. The composition of claim 1 wherein said first active agent is incorporated in said solid nano-spheres and further comprising a second active agent encapsulated in said pH sensitive or salt sensitive matrix material wherein said pH sensitive or salt sensitive matrix material releases said second active agent upon contact with a solution having a predetermined pH or predetermined salt concentration.

3. The composition according to claim 1 wherein said pH sensitive micro-sphere degrades or dissolves when said pH sensitive micro-sphere contacts a solution having a pH in the range of about 3 to about 12.

4. The composition according to claim 1 wherein said pH sensitive or salt sensitive matrix material degrades or dissolves when said pH sensitive micro-sphere contacts a solution having a pH greater than about 5.

5. The composition according to claim 1 wherein said pH sensitive or salt sensitive matrix material is a cationic pH sensitive polymer that is water insoluble at a pH above about 9 water soluble at a pH of about 7 or below.

6. The composition of claim 1 wherein said pH sensitive material is selected from the group consisting of:
acrylate polymers with amino substituents, acrylic acid esters, polyacrylamides, phthalate derivatives and mixtures thereof.

7. The composition of claim 1 wherein said pH sensitive material is selected from the group consisting of:
acid phthalate of carbohydrate, amylose acetate phthalate, cellulose acetate phthalate, cellulose ester phthalate, cellulose ether phthalate, hydroxy propyl cellulose phthalate, hydroxypropyl ethylcellulose phthalate, hydroxypropyl methyl cellulose phthalate, methyl cellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinyl acetate phthalate copolymer, styrene and maleic acid copolymer, gelatin, gluten, shellac, salol, keratin, keratin sandarac-tolu, ammoniated shellac, benzophenyl salicylate, cellulose acetate trimellitate, cellulose acetate blended with shellac, hydroxypropylmethyl cellulose acetate succinate, oxidized cellulose, polyacrylic acid derivative, acrylic acid and acrylic ester copolymers, methacrylic acid, methacrylic acid ester, vinyl acetate, crotonic acid copolymer and mixtures thereof.

8. The composition of claim 1 wherein a first portion of said plurality of nano-spheres are adhered to a second portion of said plurality of nano-spheres with a pH sensitive or salt sensitive matrix material.

9. The composition of claim 1 further comprising a moisture sensitive material mixed with said pH sensitive or salt sensitive material of said micro-sphere.

10. The composition of claim 9 wherein said moisture sensitive material is selected from the group consisting of polyvinyl pyrrolidone, water soluble cellulose, polyvinyl alcohol, ethylene maleic anhydride copolymer, methyl vinyl ether maleic anhydride copolymer, polyethylene oxides, polyamide, polyester, copolymers or homopolymers of acrylic acid, polyacrylic acid, polystyrene acrylic acid copolymer, starch derivatives, polyvinyl alcohol, acrylic acid copolymer, anionic polymer of methacrylic acid and methacrylate, cationic polymer having dimethyl-aminoethyl ammonium functional groups, hydroxyethyl cellulose, carboxymethyl cellulose, hydroxymethyl cellulose, carboxymethyl cellulose, hydroxypropyl carboxymethyl cellulose, hydroxypropyl methyl carboxyethyl cellulose, hydroxypropyl carboxypropyl cellulose, hydroxybutyl carboxymethyl cellulose, polysaccharide, hydrocolloid, natural gum, protein, and mixtures thereof.

11. The composition of claim 1 wherein said pH sensitive material is relatively insoluble and impermeable at the pH of the stomach and is more soluble and permeable at the pH of the small intestine and colon.

12. The composition of claim 11 wherein said pH sensitive material is selected from the group consisting of polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

13. The composition of claim 1 wherein said solid nano-spheres are formed of a wax material has a melting point in the range of between about 25 degrees C. and about 150 degrees C.

14. The composition of claim 13 wherein said wax material has a penetration point of about 1 to about 10.

15. The composition of claim 13 wherein said wax material is selected from the group consisting of:
natural wax, synthetic wax, regenerated wax, vegetable wax, animal wax, mineral wax, petroleum wax, microcrystalline wax and mixtures thereof.

16. The composition of claim 13 wherein said wax comprises one or more of carnauba wax, candelilla wax and beeswax.

17. The composition of claim 1 wherein said solid nano-spheres are formed of a fat material is selected from the group consisting of:
hydrogenated castor oil, hydrogenated vegetable oil, hard fat, glyceride, fatty acids, fatty acid derivative, lipid, steroid and mixtures thereof.

18. The composition of claim 17 wherein said glyceride is selected from the group consisting of:
triglyceride, monoglyceride, diglyceride, glyceryl monostearate, glycerol tristearate and mixtures thereof.

19. The composition of claim 17 wherein said fatty acid derivative is selected from the group consisting of:
alcohol, ester, anhydride, hydroxy fatty acid and prostaglandin.

20. The composition of claim 17 wherein said fat material is selected from the group consisting of:
lauric acid, physeteric acid, myristoleic acid, palmitoleic acid, petroselinic acid, oleic acid, isolauric acid, isomyristic acid, isopalmitic acid, isostearic acid, isoprenoid, 12-(((7′-diethylaminocoumarin-3yl)carbonyl)methylamino)-octadecanoic acid, N-[12-(((7′diethylaminocoumarin-3-yl)carbonyl)methyl-amino)octadecanoyl]-2-aminopalmitic acid, N succinyl-dioleoylphosphatidylethanol amine, palmitoyl-homocysteine, digalactosyldiglyceride, 1,2-dioleoyl-sn-glycerol; 1,2-cdipalmitoyl-sn-3 succinylglycerol; 1,3-dipalmitoyl-2-succinylglycerol and mixtures thereof.

21. The composition of claim 17 wherein said fat material is selected from the group consisting of:
phospholipid, sphingolipid, cholesterol, steroid derivative, terpene, tocopherol, stearlyamine, vitamin and mixtures thereof.

22. The composition of claim 21 wherein said phospholipid comprises:
phosphatidic acid, phosphatidyl choline, phosphatidyl ethanolamine, phosphatidylglycerol, phosphatidylserine, phosphatidylinositol, lysophosphatidyl derivative, cardiolipin, beta-acyl-y-alkyl phospholipid, phosphatidylcholines, dioleoylphosphatidylcholine, dimyristoylphosphatidylcholine, dipentadecanoylphosphatidylcholine, dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphatidylcholine (DLPC), phosphatidylethanolamine, dioleoylphosphatidylethanolamine, 1-hexadecyl-2-palmitoylglycerophosphoethanolamine, synthetic phospholipids and mixtures thereof.

23. The composition of claim 21 wherein said steroid is selected from the group consisting of:
cholesterol, cholesterol sulfate, cholesterol hemisuccinate, 6-(5-cholesterol 3 beta-yloxy)hexyl6-amino-6-deoxy-1-thio-alpha-D-galactopyranoside, 6-(5-cholesten-3 beta-tloxy)hexyl-6-amino-6-deoxyl-1-thio-alpha-D mannopyranoside, cholesteryl(4′-trimethyl 35 ammonio)butanoate and mixtures thereof.

24. The composition of claim 1, wherein said micro-sphere further comprises a water sensitive material is selected from the group consisting of:
natural oligomer, synthetic oligomer, natural polymer, synthetic polymer and copolymer, starch, starch derivative, oligosaccharide, polysaccharide, hydrocolloid, natural gum, protein, cellulose, cellulose derivative and mixtures thereof.

25. The composition of claim 1 further comprising a bioadhesive material incorporated into said solid nano-sphere or said micro-sphere or in both said nano-sphere and said micro-sphere.

26. The composition of claim 25 wherein said bioadhesive material is a bioadhesive polymer.

27. The composition of claim 26 wherein said bioadhesive polymer is selected from the group consisting of polyhyaluronic acids, casein, gelatin, glutin, polyanhydrides, polyacrylic acid, alginate, chitosan, poly(methyl methacrylates), poly(ethyl methacrylates), poly(butyl methacrylate), poly(isobutyl methacrylate), poly(hexl methacrylate), poly(isodecl methacrylate), poly(lauryl methacrylate), poly(phenyl methacrylate), poly(methyl acrylate), poly(isopropyl acrylate), poly(isobutyl acrylate), poly(octadecl acrylate) and poly(fumaric-co-sebacic) acid.

28. The composition of claim 1 wherein said nano-sphere further comprises a ligand.

29. The composition of claim 1 wherein said nano-sphere further comprises a targeting material selected from the group comprising lectin viral protein, bacterial protein, monoclonal antibody, antibody fragment, hormone, cell-adhesion molecule, saccharide, drug and neurotransmitter.

30. The composition of claim 1 wherein said first active agent is selected from the group consisting of: an oligonucleotide, oligoribonucleotide, polynucleotide, polyribonucleotide, gene, messenger ribonucleic acid, and ribozyme.

31. The composition of claim 1 wherein said nano-spheres further comprise a cationic surface active agent, anionic surface active agent, a nonionic surface active agent or a zwitterionic surface active agent.

32. The composition of claim 1 wherein said micro-sphere has a size of from about 20 to about 100 microns.

33. The composition according to claim 1 wherein each of said nano-spheres has an average size of about 0.01 to about 5 microns.

34. The composition according to claim 1 wherein said first pharmaceutical active agent is incorporated in said micro-sphere and said nano-spheres and said pH or salt sensitive material upon contact with said solution releases said first pharmaceutical agent to provide a burst and said first pharmaceutical agent is released continuously thereafter for an extended period of time.

35. The composition according to claim 1 wherein the extended period of time is in the range of a day to a period of a few weeks.

36. The composition according to claim 2 wherein upon contact with said solution releases said second pharmaceutical agent to provide a burst and said first pharmaceutical agent is released continuously thereafter for an extended period of time.

37. The composition of claim 1 wherein the composition is formulated in an oral product.

38. The composition according to claim 37 wherein the extended period of time is in the range of a day to a period of a few weeks.

39. An article formed of the composition of claim 1.

40. A composition comprising in combination with a and physiologically acceptable carrier a controlled release composition comprising: a plurality of solid nano-spheres, said plurality of solid nano-spheres being encapsulated in a pH sensitive or salt sensitive micro-sphere, said pH sensitive or salt sensitive micro-sphere is formed of a pH sensitive or salt sensitive matrix material, and a first pharmaceutical active agent incorporated into said solid nano-spheres or said microsphere or in both said solid nano-sphere and said micro-sphere, in a quality sufficient upon administration in a single or multiple does regimen to a mammal to release said first pharmaceutical active agent, wherein said first active agent is a nucleic acid.

41 The composition of claim 40 wherein said composition is administered orally.

42. The composition according to claim 40 in which said dosage form is selected from the group consisting of powder, tablets, capsules, ligand, elixir and granules.

43. The composition of claim 1 wherein said first pharmaceutical active agent is delivered to the gastrointestinal tract.

44. The composition of claim 1 wherein said first active agent is delivered to the stomach and/or small intestine.

45. A method for delivering an active substance to a preselected environment; said method comprising the steps of introducing to said environment a control release composition, said control release comprising: a plurality of solid nano-spheres, said plurality of solid nano-spheres being encapsulated in a pH sensitive or salt sensitive micro-sphere, said pH sensitive or salt sensitive micro-sphere is formed of a pH sensitive or salt sensitive matrix material, and a first active agent incorporated into said solid nano-spheres or said microsphere or in both said solid nano-sphere and said micro-sphere, said first active agent comprising a nucleic acid,
wherein introducing of said composition into said environment permits degradation of said composition and release of said active agent.

46. The method of claim 45 wherein before step a. orally administering said controlled release composition.

47. The method of claim 45 wherein said environment is the stomach and/or small intestine.

48. The method of claim 45 wherein said pH sensitive micro-sphere degrades or dissolves when said pH sensitive micro-sphere contacts a solution having a pH in the range of about 3 to about 12.

49. The method of claim 45 wherein said pH sensitive material is selected from the group consisting of:
acid phthalate of carbohydrate, amylose acetate phthalate, cellulose acetate phthalate, cellulose ester phthalate, cellulose ether phthalate, hydroxy propyl cellulose phthalate, hydroxypropyl ethylcellulose phthalate, hydroxypropyl methyl cellulose phthalate, methyl cellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinyl acetate phthalate copolymer, styrene and maleic acid copolymer, gelatin, gluten, shellac, salol, keratin, keratin sandarac-tolu, ammoniated shellac, benzophenyl salicylate, cellulose acetate trimellitate, cellulose acetate blended with shellac, hydroxypropylmethyl cellulose acetate succinate, oxidized cellulose, polyacrylic acid derivative, acrylic acid and acrylic ester copolymers, methacrylic acid, methacrylic acid ester, vinyl acetate, crotonic acid copolymer and mixtures thereof.

50. The method of claim 45 wherein a first portion of said plurality of nano-spheres are adhered to a second portion of said plurality of nano-spheres with a pH sensitive or salt sensitive matrix material.

51. The method of claim 45 further comprising a moisture sensitive material mixed with said pH sensitive or salt sensitive material of said micro-sphere.

52. The method of claim 45 wherein said active agent is selected from the group consisting of: an oligonucleotide, oligoribonucleotide, polynucleotide, polyribonucleotide, gene, messenger ribonucleic acid, and ribozyme.

53. The method of claim 45 wherein said active agent is delivered to the stomach and/or small intestine.

54. The method of claim 45 wherein said active agent is delivered to the gastrointestinal tract.

55. The method of claim 45 further comprising a bioadhesive material incorporated into said solid nano-sphere or said micro-sphere or in both said nano-sphere and said micro-sphere.

56. The method of claim 45 wherein said bioadhesive material is a bioadhesive polymer.

57. The method of claim 45 wherein said nano-sphere further comprises a ligand.

58. The method of claim 45 wherein said nano-sphere comprises a targeting material selected from the group comprising lectin viral protein, bacterial protein, monoclonal antibody and antibody fragment.

59. A process for producing the multi component controlled release system comprising the steps of:
heating a hydrophobic material to a temperature above the melting point to form a melt;
dissolving or dispersing the first active agent into the melt;
dissolving or dispersing a second active agent, and the pH sensitive matrix, in the aqueous phase and heating it to above the melting temperature of the hydrophobic material;
mixing the hot melt with the aqueous phase to form a dispersion;
high shear homogenization of the dispersion at a temperature above the melting temperature until a homogeneous fine dispersion is obtained;
cooling the dispersion to ambient temperature; and
